(19) United States
(12) Patent Application Publication
(10) Pub. No.: US 2007/0016081 A1
(43) Pub. Date: Jan. 18, 2007

(54) Chroma-photon staining

(75) Inventors: Michael D. Harris, Scottsdale, AZ (US); Don J. Jordan, Jamul, CA (US)

Correspondence Address: JENNINGS, STROUSS & SALMON, P.L.C., 201 E. WASHINGTON ST., 11TH FLOOR, PHOENIX, AZ, 85004, US

(73) Assignee: GlobalMedia Group, LLC

(21) Appl. No.: 11/485,117

(22) Filed: Jul. 11, 2006

Related U.S. Application Data
(60) Provisional application No. 60/698,657, filed on Jul. 12, 2005.

(51) Int. Cl. H04N 7/18; H04N 9/64; A61B 6/00

(52) U.S. Cl. 600/476; 348/79; 348/253

(57) ABSTRACT

A novel method or technique of digitally enhancing the pre- or post-processed chrominance value(s) is disclosed. According to the present disclosure the invention provides for a method of digitally enhancing or “staining” the pre- and/or post-processed chrominance values of a live high-resolution digital video image through software or firmware, using a set of parametric controls and others, to isolate its peak threshold, intensity slope and slopes range. The technology is applicable for embedding digital signatures within a photograph, a pseudo H/E stain for pathological biopsy, enhancing a region-of-interest of a slide/specimen for presentational or educational purposes, enhancing a fingerprint in a forensics laboratory, highlighting a person or figure during security monitoring and enhancing traces on a printed circuit board during visual inspection in real-time spectroscopy, among other applications.

### CLAIM TO DOMESTIC PRIORITY

This application claims the benefit of priority of U.S. Provisional Patent Application Ser. No. 60/698,657, filed Jul. 12, 2005.

### FIELD OF THE INVENTION

The present invention relates to digitally enhancing a live image of an object using the chrominance and/or luminance values which could be received from a CMOS- or CCD-based video camera; and more specifically to digitally enhancing live images viewed through any optical or scanning inspection device such as, but not limited to, microscopes (dark or bright field), macroscopes, PCB inspection and re-work stations, medical grossing stations, telescopes, electron scopes and Atomic Force (AFM) or Scanning Probe (SPM) Microscopes and the methods of staining or highlighting live video images for use in digital microscopy and spectroscopy.

### BACKGROUND OF THE INVENTION

The use of micro- and macroscopic inspection has been plagued with either poor contrast or lack of definition of the object being viewed. As lenses and lighting techniques have been improved greatly over the past 50 years and have helped with the clarity and contrast of the subject matter, so have many doctors and scientists relied on “staining” the subject matter with fluoresces and other chemistries that respond to specific light wave lengths. This technique has been shown to improve some microscopic inspection industries, but only with still photography. It is also irreversible.

In fact, present digital microscopy and spectroscopy image enhancement and staining are limited to applying a chemical stain to a given slide and then taking a separate picture under several different light sources. After each picture is taken, each has to be copied over the top of the others so that each can be realized within the final photograph. The process can take several hours to perform to get a result only to find that the wrong color of light or stain was used during the build.

Further, in conventional RGB to YUV conversion systems, an interpolation of the red, green and blue data in the original pixel data is made in order to project color values for pixels in the sensor array that are not sensitive to that color. From the red, green and blue interpolated data, lumina and chroma values are generated. However, these methods do not take into account the different filtering and resolution requirements for lumina and chroma data. These systems do not optimize the filtering or interpolation process based on the lumina and chroma data. Thus, real-time viewing and broadcasting of stained object or specimens is not possible with any technology presently available or known in the art.

Therefore, a need exists for a staining method that allows all or discrete parts of an object or specimen under inspection to be stained without permanently altering it. Further, a need exists for the staining to permit real-time viewing and broadcasting of the stained object rather than stained snap-shots or a recorded version.

### SUMMARY OF THE INVENTION

The present invention includes a method of digitally staining an object comprising viewing a live digital image of an object, wherein the object includes a first element and a second element, and wherein the live digital image is comprised of a plurality of pixels and modifying the values of a plurality of pixels in the image, wherein the values are selected from a group consisting of chrominance values and luminescence values, and wherein the modification results in a digitally stained image, wherein the first element is stained a first color and the second element is stained a second color.

The method further includes modification of chrominance values of the pixels using parametric controls, wherein the chrominance value of a first pixel that falls into a first calculated chrominance range is modified to reflect the mean of a first 9Bloc. The method further includes modifying the chrominance value of a second pixel that falls into a second calculated chrominance range to reflect the chrominance mean of a second 9Bloc.

The method further comprises determining an edge between the first element and the second element by comparing the high and low chrominance values of the 16 pixels surrounding the 9Bloc relative to the mean of the 9Bloc, wherein when the chrominance mean of one of the surrounding pixels of the 9Bloc falls above or below a pre-calculated high or low threshold, an edge is demarcated. The method further comprises digitally staining a microscopic slide and inversing the image digitally to simulate a dark-field environment.

The present invention also includes a chrominance enhancing method or technique, comprising digitally changing the chrominance and/or luminance value(s) of either pre- or post-processed individual pixel information of a CCD or CMOS imaging sensor through software and/or firmware calls. The method also includes real-time video that is either monochromatic or polychromatic. The present invention also includes a method of enhancing a live video image with respects to an image's individual R, G and B pixel values, thereby obtaining a modified outline of a subject displayed on a computer monitor.

The present invention also includes a method using transcoded RGB chroma values into YUV color space for the purpose of controlling the luminance and chrominance values independently by selecting the high and low chroma values based on a single selected pixel. This method also includes using an image's YUV color space in using the luminance, chrominance and alpha information to increase or decrease their values to simulate a chemical stain, using parametric type controls. Finally, the present invention includes a method of applying a minimum of six digital stains to a live digital video source for reasons of spectroscopy observation and study.

### BRIEF DESCRIPTION OF THE FIGURES

FIG. 1 illustrates a real-time image staining apparatus according to one embodiment of the present disclosure.

FIG. 2A is an example of an RGB Bayer Pattern.

FIG. 2B is a Bayer Pattern example of a chroma filter according to one embodiment of the present disclosure.

FIG. 3 is a Bayer Pattern example of an edge filter according to one embodiment of the present disclosure.

FIG. 4 illustrates the parametric stain point according to the present disclosure.

FIG. 5 is a flowchart illustrating the staining method according to the present disclosure.

FIG. 6 is a photograph of a tri-stain using the CPS technique in RGB color space of a Printed Circuit Board.

FIG. 7 is a photograph showing a live sample using the CPS technique in RGB color space, of a liver cell at a microscopic power of 100×.

FIG. 8 is a photograph showing a regional stain isolating a region-of-interest within a pre-H/E stained tissue sample at a microscopic power of 250×.

### DETAILED DESCRIPTION

The present disclosure describes a chrominance or luminance enhancing method or technique comprising of digitally changing the chrominance and/or luminance values of either pre- or post-processed “live” individual pixel information of a CCD or CMOS imaging sensor through software or firmware. This can also be described as a method of enhancing a live video image with respect to the image's individual R, G, and B (Red, Green, Blue) pixel values, thereby obtaining a modified outline of the subject displayed on a computer monitor or other types of image viewing devices known in the art.

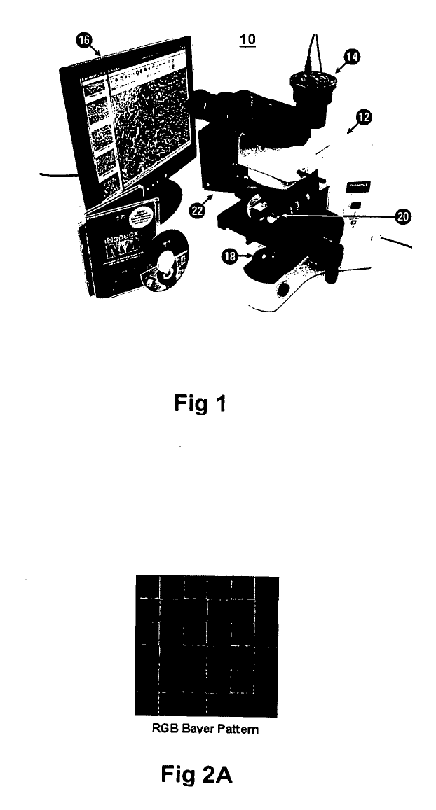

FIG. 1 illustrates one example of an apparatus suitable to carrying out the disclosed method. Digital Staining Device 10 includes microscope 12 and camera 14. Camera 14 can be any type of CCD or CMOS imaging sensor known in the art. In FIG. 1, microscope 12 is a color CCD video-based microscope system that allows the user to view small objects on video monitor 16 through camera 14. Other suitable viewing systems can be used.

According to FIG. 1, light 18 is used to provide illumination for viewing the target object 20 on the video monitor 16. Light 18 can be natural light, artificial light, such as overhead room lights, or can be a light source particular to the staining apparatus, such an LED light, Raman fixed-focus laser or a standard halogen microscope light aperture. According to FIG. 1, video monitor 16 is a computer monitor connected to computer 22. Computer 22 runs the software or firmware that digitally changes the chrominance and/or luminance values of either pre- or post-processed individual pixel information received from camera 14.

Digital staining device 10 is capable of live, stained inspection methods in the applications of semiconductor, printed circuit boards, electronics, tab and wire bonding, hybrid circuit, metal works, quality control and textiles. Digital staining device 10 can also be any optical or scanning inspection device such as, but not limited to, microscopes (dark or bright field), macroscopes, printed circuit board inspection and re-work stations, medical grossing stations, telescopes, Electron, Atomic Force (AFM) or Scanning Probe (SPM) Microscopes and the methods of staining or highlighting live video images for use in digital microscopy, histogroscopy and spectroscopy.

According to this invention, a chemical, florescent or other stain can be simulated when the YUV color spaces using the luminance, chrominance and alpha information to increase or decrease its values based on the pre-calculated parametric controls. This invention can further be used to digitally stain a microscope slide and then digitally inversing the image to highlight a region of interest or completely turn deselected pixels to black in order to simulate a dark-field environment. As shown in FIG. 6, this invention is also particularly useful in enhancing traces of a Ball Grid Array (BGA) component on a printed circuit board during visual inspection for real-time spectroscopy and quality control.

Digital staining device 10 is also capable of producing “live” or real-time staining of moving objects such as small organisms, single-celled organisms, cell tissue and other biological specimens. Specifically, the present invention discloses a method of digitally staining an object comprising: viewing a live digital image of an object, wherein the object includes a first element and a second element or more, and wherein the live digital image is comprised of a plurality of pixels; and modifying the values of a plurality of pixels in the image, wherein the values are selected from a group consisting of chrominance values and luminance values, and wherein the modification results in a digitally stained image, wherein the first element is stained a first color and the second element is stained a second color, and the third element is stained a third color and so on.

The present invention is also useful in detecting embedded digital signatures within a photograph, enhancing a fingerprint in a forensics laboratory, or highlighting a particular person or figure during security monitoring. According to the present invention, the method described above will hereinafter be referred to as Chroma-Photon Staining or CPS. It should be noted that the following explanation uses 8-bit values for the RGB and YUV color components, by way of example only. However, the CPS technique is not limited to 8-bit values.

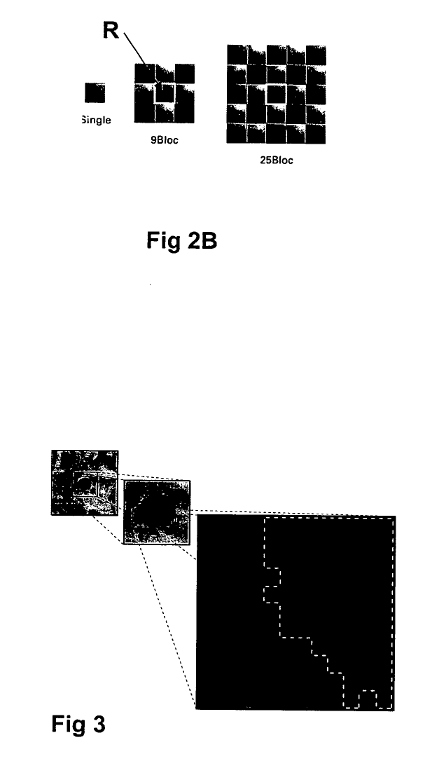

The imaging sensors, such as camera 14, are usually arranged in Red, Green, Blue (RGB) format, and therefore data is obtained from these video sensors in RGB format. However, RGB format alone is inadequate for carrying out the method according to the present disclosure, in that RGB format does not permit separating the chrominance and luminance properties. Therefore, the present invention ultimately utilizes the YUV color space format. YUV color space allows for separating the chrominance and luminance properties of RGB format. Thus, according to the invention, the RGB values are trans-coded into YUV color space using an algorithm for the purpose of controlling the chrominance and luminance values independently. This is accomplished by selecting the high and low chroma values based on a 9Bloc (defined below) of a single selected pixel. FIG. 2A illustrates an RGB Bayer Pattern, while FIG. 2B illustrates the chroma filter by way of a Bayer Pattern example.

As shown in FIG. 2B, the chrominance values of the pixels a re-modified using real-time parametric controls, wherein the chrominance value of a first pixel that falls into a first calculated chrominance range is modified to reflect the mean of a 9Bloc of pixels. According to this invention a 9Bloc is a union of nine pixels, three high and three wide. The center pixel is the reference (or defining) pixel and the surrounding dihedral group of the neighboring 8 pixels completes the 9Bloc. As shown in FIG. 2B, and by way of example only, R is the center pixel and the reference pixel.

In one embodiment, the method further demarcates an edge between the first element and the second element by comparing the high and low chrominance values of the 16 pixels surrounding the 9Bloc—in other words, the outer edge of a pixel block that is 25 pixels (five high and five wide), hereinafter denoted as a 25Bloc, with the mean of the 9Bloc (or the new value of the reference pixel). When the chrominance mean of one of the surrounding pixels rises above or falls below a pre-calculated high or low threshold relative to the mean of the 9Bloc, an edge is demarcated. FIG. 3 illustrates one example of the edge filter.

As shown in FIG. 3, the CPS edge filter looks for edges by comparing the high and the low chrominance values of the adjacent three pixels, the adjacent two pixels and the adjacent one pixel of the selected reference pixel (9Bloc). This is very different from the Canny and Di Zenzo algorithms as they compute the magnitude and direction of the gradient (strength and orientation for the compass operator) followed by non-maximal suppression to extract the edges. The CPS technique uses levels or magnitudes of color relative to the mean of the selected 9Bloc chosen to stain. The CPS filter simply looks beyond the 9Bloc in each direction. First one pixel out, then two, and then three in each direction, calculating the mean each time. This feature can be turned off or on within the filter. This technique can keep the stain concentrated to selected areas of the object and instead of the entire viewing scene. The example in FIG. 3 is demonstrative of this feature of the invention.

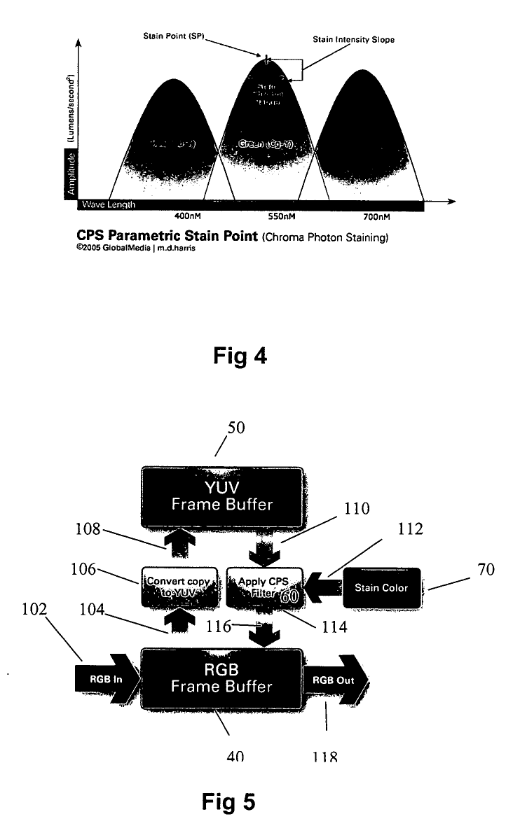

FIG. 4 illustrates the parametric staining point, stain intensity and stain chroma range according to the present disclosure. The stain point is the 9Bloc selected by the user for staining, the stain intensity is the luminance value above the selected 9Bloc and the stain chroma range is bandwidth of the chrominance value relative to the 9Bloc selected.

CPS allows the spectroscopic stain maker to work in real-time with the live image which may or may not be chemically stained. Controlling the lighting environment is important for the CPS technique to have favorable results. Keeping a consistent “flood” of light and light temperature assists in obtaining consistent staining.

To better control the color conversion of the data from a camera sensor, the present data is to convert or “transcode” the Red, Green and Blue (RGB) data into YUV 4:4:4 color space. As shown in FIG. 4, Blue also can be expressed as Cb-Y; Green as Cg-Y; and Red as Cr-Y.

Instead of each pixel having three color values, RGB, the color information is transcoded to CbCr color which is the U and V values. According to the present disclosure:
U=Cblue [1]
V=Cred [2]

The YUV conversion is accomplished according to the following equations:
Y=0.257R+0.504G+0.098B+16 [3]
U=−0.148R−0.291G+0.439B+128 [4]
V=0.439R−0.368G−0.071B+128 [5]

According to the present disclosure, the Y is the luma value. In one embodiment of the present disclosure, the user controls this feature independently from the color values, so the entire equation is:
Y=CbCr [6]

Green color is calculated by subtracting Cr from Cb, and the equation is:
Cg=Cb−Cr [7]

All notations are in hex values of FF(h) or less for 8 bit camera sensors and 400(h) for 10 bit camera sensor. The CPS technique does not involve any sub-sampling, thus, there is no color loss during the transcoding. Further, there is no compression.

Another issue with camera sensors and the CPS technique is that its accuracy is subject to the data received. High-grade CCDs have much higher dynamic range and signal to noise ration (SNR) than that of consumer grade CCDs or CMOS sensors. Sensors with 8 bit outputs will have far less contrast and DR than that of a 10 or 12 bit sensor. Other sensor issues such as temporal noise, fixed pattern noise, dark current and low pass filtering come into play with the pre-processed sensor data also.

With this in mind, a low-grade camera is less preferred then that of a high-grade for carrying out the CPS technique. However, the present disclosure envisions taking the particular conditions of the camera into consideration when using the CPS method. Therefore, the implementation of the present disclosure envisions using a high-grade CCD and a 10 or 12 bit sensor for optimal results. Dynamic Range (DR) quantifies the ability of a sensor to adequately image both highlights and dark shadows in a scene. It is defined as the ratio of the largest non-saturating input signal to the smallest detectable input signal. DR is a major factor of contrast and depth of field.

Referring back to FIG. 2B, when the user clicks the mouse in the video frame or otherwise designates a reference pixel, the RGB values of the pixel under the pointer and of the eight adjacent pixels around the point are averaged to produce a single RGB sample pixel. In FIG. 2B, and by way of example only, R is the reference pixel and would be the pixel chosen by the pointer. Thus, according to the example in FIG. 2B, R=Reference Pixel.

Modification or filtering of the 9Bloc of pixels is accomplished by averaging the four Green and four Blue pixel values with the one R value and arriving at certain averaged value, here equal to a value “A.” Therefore, according to FIG. 2B:
A=mean 9Bloc=mean(4G and 4B and 1R) [8]

Thus, A is also the new value of the reference pixel. As further shown in FIG. 2B, the 25 block of pixels is then modified by first averaging the outside sixteen pixels. In the example in FIG. 2, this is accomplished by averaging the eight Green and eight Red values to arrive at a certain average value, here equal to a value “B.” Thus, according to FIG. 2B:
B=mean of the outside 16 pixels of the 25Bloc=mean(8G and 8R) [9]

The modification of the 25Bloc is then accomplished by the following equation:
C=mean(A and B) [10]

The reference pixel contains three, 8-bit values, ranged 0 to 255 for each red, green and blue component. These RGB values are then transformed into YUV color space using the equations:
Y=0.257R+0.504G+0.098B+16 [11]
U=−0.148R−0.291G+0.439B+128 [12]
V=0.439R−0.368G−0.071B+128 [13]

The final 8-bit YUV component values represent the key pixel that is then used as the mean for the current bandwidth ranges. The bandwidth is an 8-bit value that represents the deviation above and below a component key pixel value that determines the bandwidth range for a color component. There are two bandwidth values used by the CPS technique: the first is applied to the luminance component (Y) of the key pixel while the second is applied to both chrominance components (U and V) of the key pixel. These values are saturated to the 0 and 255 levels to avoid overflow and underflow wrap-around problems. Thus:

Y_high = Y_key + luma_bandwidth;

If(Y_high > 255)

Y_high = 255;

Y_low = Y_key − luma_bandwidth;
If(Y_low < 0)

Y_low = 0;

| | |
|---|---|
| U_high = U_key + chroma_bandwidth; | [14] |
| If(U_high > 255) | |

U_High = 255;

U_low = U_key − chroma_bandwidth;
If(U_low < 0)

U_low = 0;

V_high = V_key + chroma_bandwidth;
If(V_high > 255)

V_high = 255;

V_low = V_key − chroma_bandwidth;
If(V_low < 0)

V_low = 0;

Referring now to FIG. 5, RGB enters the RGB frame buffer 40 in step 102. The RGB Frame Buffer is a very large area of memory within the host computer that is used to hold the frame for display. A copy is then made of an incoming RGB video frame in step 104. This copy is then transformed into a YUV 4:4:4 color space format using equations [11], [12] and [13] in step 106, and is stored in the YUV frame buffer 50 in step 108. The video frame is stored in the YUV Frame buffer long enough to hand off to a CPS filter 60 in step 110 and blended with a staining color 70 of the user's choice, in step 112.

Next, the CPS technique is applied in step 114. In step 114, each YUV component of each pixel in the copied video frame is checked against the high and low bandwidth ranges calculated above. In step 114, if all YUV components of a pixel fall within the bandwidth ranges, then the corresponding pixel in the original RGB frame is stained. The stain color is an RGB value that is alpha blended with the RGB value of the pixel being stained.

The alpha blend value ranges from 0.0 to 1.0. The alpha blending formula is the standard used by most production switchers or video mixers known in the art. Thus, alpha blending is accomplished according to the following:

If ((copy_pixel_Y <= Y_high) &&
(copy_pixel_Y >= Y_low) &&

| (copy_pixel_U <= U_high) && | |
|---|---|
| (copy_pixel_U >= U_low) && | |
| (copy_pixel_V <= V_high) && | |
| (copy_pixel_V >= V_low)) | |
| {orig_pixel_R = alpha * stain_R + (1.0 − alpha) * | [15] |
| orig_pixel_R; | |
| orig_pixel_G = alpha * stain_G + (1.0 − alpha) * | |
| orig_pixel_G; | |
| orig_pixel_B = alpha * stain_B + (1.0 − alpha) * | |
| orig_pixel_B;} | |

In step 116, the stained RGB pixels enter the RGB frame buffer, and in step 118, the stained RGB image is produced.

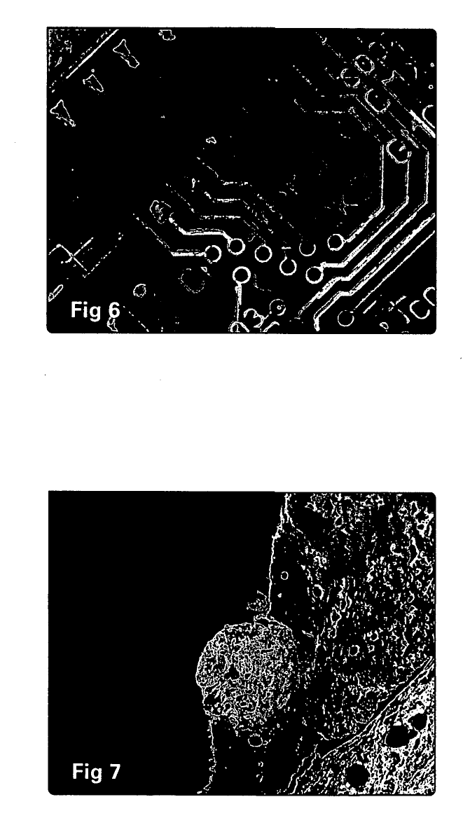

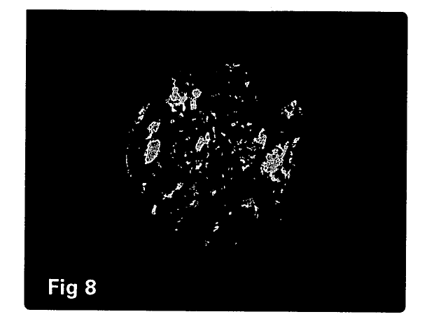

Finally, multiple stains, each with their own key pixels, bandwidths and stain colors, may be applied to the same video frame in order to demarcate elements of the target object. FIG. 6 illustrates one application of the chroma-photon staining method. FIG. 6 is a photograph of a tri-stain using the CPS technique in RGB color space of a Printed Circuit Board. FIG. 7 shows a second application of the chroma-photon staining method. FIG. 7 is a photograph showing a live sample of a tri-stain using the CPS technique in RGB color space, of a liver cell at a microscopic power of 100× magnification. FIG. 8 is a regional stain isolating out a region-of-interest within a pre-H/E (Hematoxylin & Eosin or H&E) stained tissue sample magnified at 250×.

Therefore, the present disclosure and invention provide for an advantageous staining method that allows all or discrete parts of an object or specimen under inspection to be stained without permanently altering it. Further, the present disclosure and invention permits real-time viewing and broadcasting of the stained object which is not possible with any technology presently available or known in the art. The ability of the present method to allow real-time viewing and broadcasting, versus snap shots or video recordings that are the only options currently available, provides for a superior ability to manipulate the staining of the object or specimen, including the ability for two viewers in remote geographical areas to both manipulate the staining and viewing of the object or specimen in real-time. This novel method then provides users enhanced ability to exchange ideas and communicate more efficiently and effectively about the object or specimen that is the subject matter of the chroma-photon staining.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of a preferred embodiment and best mode of the invention known to the applicant at this time of filing the application has been presented and is intended for the purposes of illustration and description. It is not intended to be exhaustive nor limit the invention to the precise form disclosed and many modifications and variations are possible in the light of the above teachings. The embodiment was chosen and described in order to best explain the principles of the invention and its practical application and to enable others skilled in the art to best utilize the invention in various embodiments and with various modifications as are suited to the particular use contemplated. Therefore, it is intended that the invention not be limited to the particular embodiments disclosed for carrying out the invention.

What is claimed:

1. A method of digitally staining an object comprising:
viewing a digital image of an object, wherein the object includes a first element and a second element, and wherein the live digital image is comprised of a plurality of pixels; and
modifying the values of a plurality of pixels in the image, wherein the values are selected from a group consisting of chrominance values and luminescence values, and wherein the modification results in a digitally stained image, wherein the first element is stained a first color and the second element is stained a second color.

2. The method of claim 1, wherein the chrominance values of the pixels are modified using parametric controls, wherein the chrominance value of a first pixel that falls into a first calculated chrominance range is modified to reflect the mean of a first 9Bloc.

3. The method of claim 2, wherein the chrominance value of a second pixel that falls into a second calculated chrominance range is modified to reflect the chrominance mean of a second 9Bloc.

4. The method of claim 1, further comprising determining an edge between the first element and the second element by comparing the high and low chrominance values of the 16 pixels surrounding the 9Bloc relative to the mean of the 9Bloc, wherein when the chrominance mean of one of the surrounding pixels of the 9Bloc falls above or below a pre-calculated high or low threshold, an edge is demarcated.

5. The method of claim 1, further comprising digitally staining a microscopic slide and inversing the image digitally to simulate a dark-field environment.

6. The method of claim 1, wherein the pixels in the image include pre-processed pixel information from an imaging sensor.

7. The method of claim 1, wherein the pixels in the image include post-processed pixel information from an imaging sensor.

8. The method of claim 1, wherein the imaging sensor is selected from a group consisting of a CCD imaging sensor, a CMOS imaging sensor or any optical scanning array sensor.

9. The method of claim 1, further including transcoding the RGB values of the pixels to YUV values.

10. The method of claim 9, wherein the RGB values are transcoded to YUV values using an algorithm, wherein the algorithm includes
Y=0.257R+0.504G+0.098B+16;
U=−0.148R−0.291G+0.439B+128;
and
V=0.439R−0.368G−0.071B+128.

11. The method of claim 1, wherein the digital image is viewed in real-time video.

12. The method of claim 11, wherein the real-time video is selected from a group consisting of monochromatic and polychromatic.

13. The method of claim 1, further including selecting high and low chrominance values based on a reference pixel.

14. The method of claim 1, further including controlling the luminance values and controlling the chrominance values, wherein the luminance values can be controlled independently of the chrominance values.

15. A computer-readable storage medium containing computer executable code for instructing a computer to perform the steps of:
copying an image comprised of a first element and a second element, wherein the first element and second element are each comprised of a plurality of pixels and each pixel has an RGB value;
transcoding the RGB values of the plurality of pixels into YUV values; and
modifying the YUV values of the plurality of pixels in the image, wherein the YUV values are selected from a group consisting of chrominance values and luminescence values, and wherein the modification results in a digitally stained image, wherein the first element is stained a first color and the second element is stained a second color.

16. The computer readable storage medium of claim 15, further including displaying the digitally stained image on a computer monitor.

17. The computer readable storage medium of claim 15, wherein the RGB values are transcoded to YUV values using an algorithm, wherein the algorithm includes
Y=0.257R+0.504G+0.098B+16;
U=−0.148R−0.291G+0.439B+128;
and
V=0.439R−0.368G−0.071B+128.

18. The computer readable storage medium of claim 15, further including alpha blending the RGB value of a stain color with the RGB of one of the plurality of pixels.

19. The computer readable storage medium of claim 18, wherein the stain color and the pixel are alpha blended using an algorithm, wherein the algorithm includes
If ((copy_pixel_Y <= Y_high) && (copy_pixel_Y >= Y_low) &&
(copy_pixel_U <= U_high) && (copy_pixel_U >= U_low) &&
(copy_pixel_V <= V_high) && (copy_pixel_V >= V_low))
{orig_pixel_R = alpha * stain_R + (1.0 − alpha) * orig_pixel_R;
orig_pixel_G = alpha * stain_G + (1.0 − alpha) * orig_pixel_G;
orig_pixel_B = alpha * stain_B + (1.0 − alpha) * orig_pixel_B;}

20. A method of enhancing a live video image:
viewing a live digital image of an object, wherein the object includes a first element and a second element, and wherein the live digital image is comprised of a plurality of pixels;
modifying the values of a plurality of pixels in the image, wherein the values are selected from a group consisting of chrominance values and luminescence values, and wherein the modification results in a digitally stained image, wherein the first element is stained a first color and the second element is stained a second color; and
allowing movement of the object, wherein the first element remains stained the first color and the second element remains stained the second color while the object is moving.
